(19) United States
(12) Patent Application Publication
(10) Pub. No.: US 2022/0288404 A1
(43) Pub. Date: Sep. 15, 2022

(54) METHODS FOR APPLYING TUMOR TREATING FIELDS IN COMBINATION WITH CANCER TREATING THERAPEUTICS

(71) Applicant: THE BOARD OF REGENTS OF THE UNIVERSITY OF TEXAS SYSTEM, Austin, TX (US)

(72) Inventors: Michael D. STORY, Dallas, TX (US); Narasimha Kumar KARANAM, Dallas, TX (US)

(73) Assignee: THE BOARD OF REGENTS OF THE UNIVERSITY OF TEXAS SYSTEM, Austin, TX (US)

(21) Appl. No.: 17/693,108

(22) Filed: Mar. 11, 2022

Related U.S. Application Data
(60) Provisional application No. 63/160,692, filed on Mar. 12, 2021.

(51) Int. Cl. A61N 1/40; A61K 45/06; A61P 35/00

(52) U.S. Cl. CPC A61N 1/40; A61K 45/06; A61P 35/00

(57) ABSTRACT

A method of treating a tumor in a subject, the method comprises delivering an ATR inhibitor to the tumor, and applying a tumor treating field to the tumor at a frequency between approximately 50 kHz and approximately 1,000 kHz.

### CROSS-REFERENCE TO RELATED APPLICATIONS

This application claims priority to the U.S. Provisional Application No. 63/160,692, filed Mar. 12, 2021, which is incorporated herein by reference.

### BACKGROUND

Tumor treating fields (TTFields) are low intensity alternating electric fields within the intermediate frequency range, which may be used to treat tumors as described in U.S. Pat. No. 7,565,205. TTFields are induced non-invasively into a region of interest by transducers placed directly on the patient's body and applying AC voltages between the transducers. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

There are certain options, such as surgical resection, chemotherapy, radiation therapy, and immunotherapy, available for cancer treatment.

### SUMMARY OF THE INVENTION

One aspect of the invention is directed to a method of treating a tumor in a subject, the method comprises: delivering an ATR inhibitor to the tumor; and applying a tumor treating field to the tumor at a frequency between approximately 50 kHz and approximately 1,000 kHz.

One aspect of the invention is directed to a method of preventing/reducing proliferation of a cell, the method comprises: delivering at least one DNA replication stress inducing agent to the cell, wherein the DNA replication stress inducing agent comprises an ATR inhibitor; and applying a tumor treating field to the cell at a frequency between approximately 50 kHz and approximately 1,000 kHz.

One aspect of the invention is directed to a method of treating a tumor in a subject, comprising: delivering at least two DNA replication stress inducing agents to the tumor, wherein at least one of the DNA replication stress inducing agents comprises an ATR inhibitor; delivering a radiation therapy to the tumor; and applying tumor treating fields to the tumor at a frequency between approximately 50 kHz and approximately 1,000 kHz.

### BRIEF DESCRIPTION OF THE DRAWINGS

FIG. 1 depicts example approaches for treating a tumor in a subject in accordance with exemplary embodiments of the present disclosure.

FIG. 2 depicts an example method of treating a tumor in a subject.

FIG. 3 depicts example effects of TTFields, Olaparib, radiation, and a combination thereof on pancreatic cancer cells.

FIG. 4 depicts example effects of TTFields, AZD6738, radiation, and a combination thereof on non-small cell lung cancer cells (NSCLC).

FIG. 5 depicts example effects of TTFields, AZD6738, radiation, and a combination thereof on pancreatic cancer cells.

FIG. 6 depicts example effects of TTFields, Cisplatin, Etoposide, and a combination thereof on NSCLC.

FIG. 7 depicts example effects of TTFields, Irinotecan, radiation, and a combination thereof on NSCLC.

FIG. 8 depicts example effects of TTFields, Irinotecan, radiation, and a combination thereof on pancreatic cancer cells.

FIG. 9 depicts example effects of TTFields, 5-FU, radiation, and a combination thereof on NSCLC.

FIG. 10 depicts example effects of TTFields, 5-FU, radiation, and a combination thereof on pancreatic cancer cells.

FIG. 11 depicts one example of an apparatus to apply TTFields with modulated electric fields to a subject's body.

FIG. 12 depicts an example computer apparatus.

### DESCRIPTION OF EMBODIMENTS

Techniques for treating a tumor in a subject are disclosed. The present disclosure relates to Tumor Treating Fields (TTFields) that may induce synergistic cell killing via the disruption of DNA damage response, enhanced DNA replication stress and DNA replication fork collapse.

The disclosed TTFields is a physical modality therapy, which can be used for the treatment of recurrent glioblastoma (GBM) as monotherapy, for diagnosed GBM in combination with temozolomide, and for unresectable locally advanced or metastatic malignant pleural mesothelioma (MPM) in combination with pemetrexed and platinum-based chemotherapy, among other cancers. The disclosed TTFields can be low-intensity, intermediate frequency, alternating electric fields, which can be loco-regionally applied to tumor sites using non-invasive arrays.

The disclosed TTFields can reduce tumor cells through the disruption of mitosis. Furthermore, the disclosed TTFields can affect DNA damage repair and replication stress pathways of tumor cells. For example, the disclosed TTFields treatment can decrease Fanconi Anemia (FA) pathway signaling proteins by impairing irradiation (IR)-induced DNA damage repair processes. The length of newly replicated DNA can be slowed as a function of TTFields exposure time, and TTFields can increase R-loop formation, which indicates that TTFields induced replication stress. The disclosed TTFields can increase the sensitivity of chemotherapy agents that target and increase replication stress in novel combination therapy options.

As recognized by the inventor, the dual specter of poor prognosis and unfavorable therapeutic index of cancer disease calls for novel therapeutic interventions and combined therapy modality options to improve overall survival rates in patients.

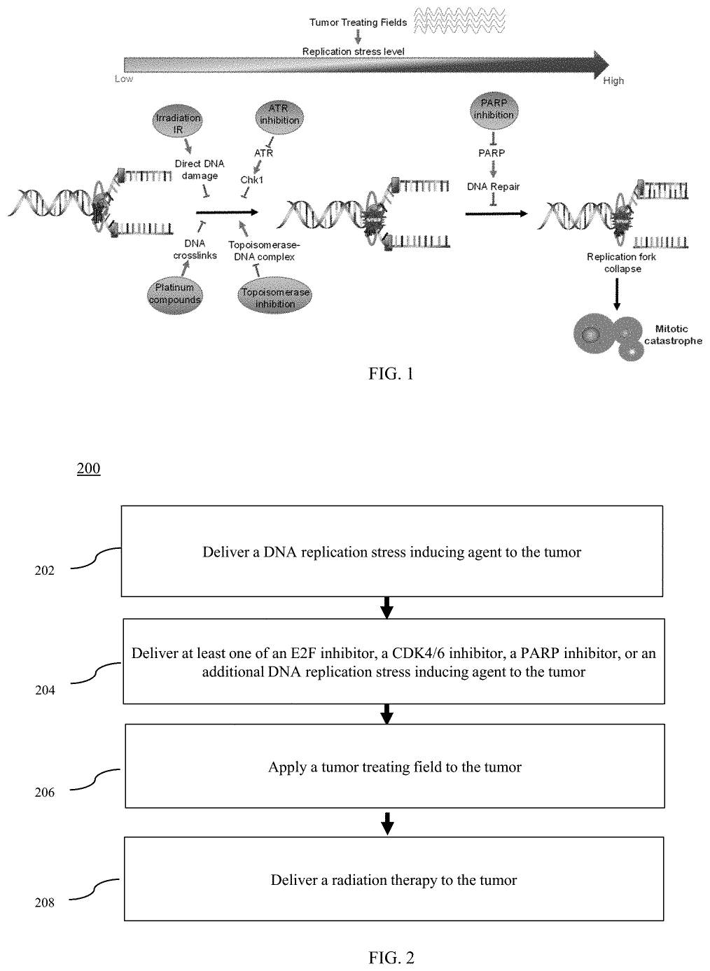

FIG. 1 depicts example inventive approaches for treating a tumor in a subject in accordance with exemplary embodiments of the present disclosure. Each of these inhibitors may target, for example, DNA repair, DNA replication, DNA crosslinks, DNA complex, or cell cycle regulation in order to increase replication stress. For example, ATR inhibitors may be introduced to tumor cells or tissue for inhibiting the cell cycle checkpoint upon DNA damages. Ionizing radiation (IR) can be applied to the tumor cell or tissue for breaking DNA strands. Platinum agents can be delivered to the tumor cell or tissue for inducing intra-strand crosslinks that can inhibit DNA replication. Repair of crosslinks can be downregulated by TTFields. PARP inhibitors can be applied to tumor cells or tissue for the inhibition of replication fork maintenance. Topoisomerase inhibitors can be delivered to tumor cells or tissue for the inhibition of DNA replication and chromosome condensation.

The disclosed combination therapy options using chemotherapeutic agents can synergistically increase replication stress in combination with TTFields. The disclosed chemotherapeutic agents can target replication stress, which can be the primary cause of genome instability. Cancer cells can maintain unrestrained proliferation by keeping low to mild levels of replication stress with defective DNA damage response (DDR) and loss of cell cycle checkpoints. Normal cells can maintain genome stability through the coordinated actions of DDR and cell cycle checkpoints. Defects in DDR and mild to low levels of replication stress are unique to cancer cells and, therefore, can be therapeutically exploited. To exploit replication stress, the disclosed TTFields can be combined with the disclosed chemotherapeutic agents, which can also cause replication stress at several key steps.

FIG. 2 depicts an example method 100 of treating a tumor in a subject. At step 202, a DNA replication stress inducing agent can be delivered to tumor cells or tissue. The DNA replication stress inducing agent can include at least one of at least one DNA misincorporation/modifying chemo agent. As an example, the DNA misincorporation/modifying chemo agent may be a platinum compound, an alkylating agent, a weel inhibitor, a Chkl inhibitor, a thymidylate synthase inhibitor, a ribonucleotide reductase inhibitor, Topoisomerase I inhibitor, Topoisomerase II inhibitors, a maternal embryonic leucine zipper kinase (MELK) inhibitor, a NEDD8-activating enzyme (NAE) inhibitor, an Ataxia telangiectasia Rad3-related protein (ATR) inhibitor, or a combination thereof.

In one example, the DNA replication stress inducing agent may be an ATR inhibitor. The ATR inhibitor can include at least one of Schisandrin B, Nu6027, Dactolisib, EPT-46464, VE-821, AZ20, Berzosertib, Torin-2, Ceralasertib (AZD6738), Tetrahydropyrazolo [1,5-a]pyrazines, Azabenzimidazoles, Gartisertib, (M4344 or VX-803), Bayl895344 (Elimsuretib), CGK 733, RP-3500, ATR-IN-4, VE-821, AZ20, ETP-46464, or ATR inhibitor 1.

In non-limiting embodiments, the ATR inhibitor may include AZD6738. AZD6738 is an ATR inhibitor that can be an essential kinase for replication checkpoint, playing an important role in safeguarding genome integrity from replication stress. In one example, Ceralasertib (AZD6738) in an amount between approximately 1 μM and approximately 50 μM can be delivered to the tumor cell, tissue, or subject. In non-limiting embodiments, Ceralasertib (AZD6738) in an amount between approximately 40 mg to approximately 240 mg can be delivered to a subject once daily for approximately 1 day to approximately 21 days.

In one example, the DNA replication stress inducing agent may be a topoisomerase inhibitor that can cause DNA strand breaks and increase replication stress with or without TTFields. The topoisomerase inhibitor may include at least one of a topoisomerase I inhibitor, a topoisomerase II inhibitor, or a combination thereof. The topoisomerase I inhibitor may include campthectin derivatives (topotecan, irinotecan, belotecan, gimatecan, silatecan), indenoisoquinoline (NSC314622, indotecan, indimitecan), phenanthridines (topovale), and indolocarbazoles (BE-13793C), SN-38, Camptothecin, Exatecan mesylate, Topotecan hydrochloride, Dxd, Betulinic acid, β-Lapachone, PNU-159682, Genz-644282, LMP744 hydrochloride, coralyne chloride, 9-amino-CPT, Namitecan, Karenitecin, CH-0793076, Edotecarin, SW044248, Exatecan, Datopotamab deruxtecan, T-2513, Podocarpusflavone A, (±)-Evodiamine, TP3011, Hycanthone, Belotecan hydrochloride, Proscillaridin A, TAS-103 dihydrochloride, Zabofloxacin, Intoplicine, Huanglongmycin N, Dxd-d5, Groenlandicine, Rebeccamycin, Intoplicine dimesylate, or a combination thereof. In non-limiting embodiments, the topoisomerase II inhibitor may include anthracyclines (doxorubicin, daunorubicin, epirubicin, idarubicin), etoposide, teniposide, dexrazoxane, novobiocin, merbarone, anthrycycline aclarubicin, Mitoxantrone, Pirarubicin, Teniposide, Bisantrene, Amsacrine, Pirarubicin Hydrochloride, Pixantrone dimaleate, Ellipticine hydrochloride, Amsacrine hydrochloride, Voreloxin Hydrochloride, Amonafide, PluriSIn #2, Gatifloxacin, Flumequine, MC-DOXHZN hydrochloride, Amrubicin, ARN-21934, Ellipticine, Pixantrone, MC-DOXHZN, CP-67804, CP-67015, Voreloxin, Elomotecan hydrochloride, Gatifloxacin mesylate, 9-Hydroxyellipticine hydrochloride, Aldoxorubicin, Hycanthone, Chloroquinoxaline sulfonamide, Proscillaridin A, Aurintricarboxylic acid, or a combination thereof.

In one example, the topoisomerase inhibitor can be etoposide that may form a ternary complex with Topoisomerase II and prevent re-ligation of DNA strands to cause DNA strand breaks and increase replication stress. Supercoiling of DNA may occur during strand separation in front of the replication and transcription sites which is relieved by topoisomerases. Failure to deal with supercoils and entanglements may result in replication fork stalling and collapse. In non-limiting embodiments, in combination with other agents, etoposide may be administered in an amount between approximately 35 mg/m² intravenous (IV) over 30 to 60 minutes once a day for 4 days to approximately 50 mg/m² IV over 30 to 60 minutes once a day for 5 days every 3 to 4 weeks. In non-limiting embodiments, the dose of etoposide in adult patients may be approximately 50 mg/m²/day to approximately 100 mg/m²/day or days 1 to 5 or 100 to 120 mg/m² on days 1, 3, and 5 every 3 to 4 weeks, with/without other inhibitors/agents.

In one example, the topoisomerase inhibitor can be irinotecan which is a semisynthetic analog of camptothecin and Topoisomerase I inhibitor. Irinotecan may trap Topoisomerase I-DNA in a ternary cleavage complex and may inhibit both the initial cleavage reaction and relegation steps. The collision of the replication fork with this complex may cause irreversible replication fork stalling and may increase replication stress. In non-limiting embodiments, irinotecan in an amount up to approximately 1.5 mg/kg per dose can be delivered to a subject daily for approximately 5 days each week for 2 weeks (i.e., one cycle of therapy), repeated every 21 days. The dose for three cycles can be approximately 10 mg/kg per dose.

In one example, the DNA replication stress inducing agent can comprise a thymidylate synthase inhibitor. In non-limiting embodiments, the thymidylate synthase inhibitor can include at least one of 5-FU or pemetrexed. For example, the 5-FU is a pyrimidine analog that may induce replication stress through the inhibition of thymidylate synthase. 5-FU can be a baseline component of first-line chemotherapy and combination therapy regimens like FULFURINOX. In non-limiting embodiments, the dose of 5-FU can be approximately 200 mg/m² body surface per day. 5-FU can be delivered to a subject through continuous intravenous infusion for approximately 3 weeks. The dose

In one example, the DNA replication stress inducing agent can comprise a ribonucleotide reductase inhibitor. In non-limiting embodiments, the ribonucleotide reductase inhibitor can include gemcitabine. The dose of gemcitabine can be approximately 1250 mg/m2 intravenously over 30 minutes. In non-limiting embodiments, the dose of gemcitabine can be delivered to a subject in combination with other agents/inhibitors. For example, approximately 1250 mg/m² can be delivered to a subject intravenously over 30 minutes on days 1 and 8 of each 21-day cycle that includes other agents/inhibitors (e.g., paclitaxel).

In one example, the DNA replication stress inducing agent can comprise a platinum compound. In non-limiting embodiments, the platinum compound can comprise at least one of cisplatin, carboplatin, oxaliplatin, dicycoplatin, lipoplatin. For example, cisplatin in an amount between approximately 20 mg/m² and approximately 120 mg/m² can be delivered to a subject.

In one example, the DNA replication stress-inducing agent can comprise an alkylating agent. In non-limiting embodiments, the alkylating agent can comprise at least one of cyclophosphamide or temozolomide.

For example, cyclophosphamide in an amount between approximately 40 mg/kg and approximately 50 mg/kg (i.e., 400-1800 mg/m²) can be delivered to a subject. The disclosed dose of cyclophosphamide can be divided over 2-5 days.

In one example, the DNA replication stress inducing agent can comprise a weel inhibitor. In non-limiting embodiments, the weel inhibitor can comprise at least one of Adavosertib-MK1775, AZD1775, or PD0166285. For example, AZD1775, in combination with other inhibitors/agents (e.g., gemcitabine) or radiation treatments, can be delivered to a subject in an amount between approximately 100 mg and approximately 175 mg. AZD1775 can be delivered to a subject once daily with/without other inhibitors/agents (e.g., gemcitabine) and radiation treatments.

In one example, the DNA replication stress inducing agent can comprise a Chk1 inhibitor. In non-limiting embodiments, the Chk1 inhibitor can comprise at least one of UCN-01, LY2606368, SAR-020106, AZD7762, or PD0166285. For example, in combination with other agents/treatments (e.g., gemcitabine), AZD7762 in an amount up to approximately 30 mg can be delivered to a subject.

In one example, the DNA replication stress inducing agent can comprise at least one of a maternal embryonic leucine zipper kinase (MELK) inhibitor (e.g., OTS167) or a NEDD8-activating enzyme (NAE) inhibitor (e.g., MLN4924). For example, OST-167 in an amount between approximately 0.5 mg to approximately 2.0 mg can be delivered to a subject.

In certain embodiments, the disclosed inhibitors can be delivered to tumor cells or tissue through various techniques. In one example, the disclosed inhibitors can be delivered to the tumor in a cocktail form. More than one inhibitor can be combined in a cocktail form and delivered to tumor cells or tissue. In non-limiting embodiments, the disclosed inhibitors can be delivered through infusion.

In certain embodiments, an additional DNA replication stress inducing agent can be delivered. In one example, the DNA replication stress inducing agent and the additional DNA replication stress inducing agent can comprise the same agent or inhibitor. In one example, the DNA replication stress inducing agent and the additional DNA replication stress inducing agent can comprise a different agent or inhibitor.

At step 204, at least one of an E2F inhibitor, a CDK4/6 inhibitor, a PARP inhibitor, or the additional DNA replication stress inducing agent may be delivered to the tumor cells or tissue.

In one example, an E2F inhibitor can be delivered to the tumor to dysregulate the E2F family of transcription factors and increase cellular vulnerabilities against agents that cause DNA damage or inhibit DNA damage repair. In non-limiting embodiments, the E2F inhibitor can be HLM006474. The E2F family of transcription factors can drive the downregulation of these specific pathways that render cells sensitive to DNA damaging agents and replication stress. The dysregulation of the E2F family of transcription factors through the disclosed inhibitors can lead to cellular vulnerabilities against agents that cause DNA damage or inhibit DNA damage repair. The application of the E2F inhibitor can include the downregulation of E2F family members that are transcription activators while specific E2F family members that are repressors are activated. This repressor E2F6, a transcription repressor, is upregulated. E2F6 ordinarily allows for the expression of BRCA1. However, as an activated repressor of transcription, BRCA1 can be downregulated.

In one example, a CDK4/6 inhibitor can be delivered to the tumor. In one example, the CDK4/6 inhibitor can include at least one of abemaciclib, palbociclib, ribociclib, or trilaciclib.

In one example, a PARP inhibitor can be delivered to the tumor to decrease the DNA repair of the tumor cells leading to cell death with or without TTFields. PARP1 protects DNA breaks by recruiting DNA repair and checkpoint proteins to the sites of damage and recruiting MRE11 for DNA end processing (required for replication restart and enhanced Chk1 activation). By introducing the PARP inhibitor, the DNA breaks of the target tumor can be enhanced. The PARP inhibitor can include Olaparib, talazoparib, veliparib, rucaparib, BYK204165, niraparib (MK-4827), niraparib (MK-4827), tosylate, or iniparib.

In certain embodiments, the tumor can include at least one of non-small cell lung cancer (NSCLC), pancreatic cancer, GBM, mesothelioma, pancreatic cancer, lung cancer, ovarian cancer, and cervical cancer. Any cancer of the head, thorax or abdomen is among the conditions that may be affected by the present disclosure. In one example, the tumor can include at least one a lung cancer cell, a breast cancer cell, a pancreatic cancer cell, a glioblastoma cell, a prostate cancer cell, a liver cancer cell, a fallopian tube cancer cell, a peritoneal cancer cell, a cervical cancer cell, a skin cancer cell, or an ovarian cancer cell.

At step 206, tumor treating fields (TTFields) can be applied to the tumor. In non-limiting embodiments, the TTFields can be applied with predetermined parameters. As an example, the TTFields can include a frequency within a frequency range from about 50 kHz to about 1000 kHz. As an example, the frequency of the tumor treating field may be between approximately 100 kHZ and approximately 500 kHZ. As an example, the frequency of the tumor treating field may be approximately 100 kHZ, approximately 150 kHZ, approximately 200 kHZ, or approximately 250 kHZ. As an example, the TTFields may include an intensity within an intensity range from about 1 V/cm to about 20 V/cm. As an example, the TTFields may include an intensity within an intensity range from about 1 V/cm to about 10 V/cm. As an example, the intensity of the tumor treating field may be between approximately 1 V/cm and approximately 4 V/cm. Other possible exemplary parameters for the TTFields may include active time, dimming time, and duty cycle (all of which may be measured in, for example, ms units), among other parameters. The parameters can be modified based on the sizes of tumor, types of tumor, subjects, or purposes of the treatment. In one example, the intensity of the tumor treating field is between approximately 1 V/cm and approximately 4 V/cm, and the frequency of the tumor treating field is between approximately 150 kHZ and approximately 250 kHZ for treating glioblastoma cancer cells.

The disclosed TTFields treatment can induce DNA damage and impair DNA damage response. The application of the disclosed TTFields can increase in γ-H2AX foci with time in cells (e.g., that were not irradiated but were exposed to TTFields). Because γ-H2AX is also an early sensor of stalled replication forks during replication stress, TTFields exposure can induce replication stress, as reduced expression of BRCA1 and other members of the Fanconi's Anemia pathway, negatively affects the repair of collapsed or stalled replication forks. Furthermore, the MCM6 and MCM10 genes, integral members of the DNA replication complex, can also be downregulated by the application of the TTFields.

In certain embodiments, the TTFields can be applied to the tumor before or after the disclosed inhibitors and/or radiation therapy is applied. In certain embodiments, the TTFields can be simultaneously applied to the target tissue with the disclosed inhibitors and/or radiation therapy. As an example, at least a portion of the applying step 206 may be performed simultaneously/concomitantly with at least a portion of the delivering step 202 and/or at least a portion of the delivering 204. In non-limiting embodiments, the disclosed agents/inhibitors can be delivered concomitant with TTFields and/or radiation therapy. As an example, at least a portion of the applying step 206 may be performed simultaneously with at least a portion of the delivering step 202 and at least a portion of the delivering step 204 and/or delivering step 208.

At step 208, radiation therapy can be delivered to the tumor. In one example, radiation therapy can be the ionizing radiation (IR) treatment. A dose of the radiation therapy can be between approximately 1 Gy to approximately 18 Gy per fraction. In one example, a dose of the radiation therapy can be between approximately 1.8 Gy to approximately 18 Gy per fraction. The dose of the radiation therapy can vary based on a target tumor type, a tumor size, a subject, or the type of the radiation therapy. For example, standard radiotherapy is 2 Gy/fraction and can last for 4-7 weeks. Stereotactic Ablative Radiotherapy (SAbR) is general 10 Gy to as high as 18 Gy per fraction for no more than 5 fractions. Fractions are delivered daily for 5 days, or if the single fractions are greater than 12, there is sometimes a rest day for normal tissue recovery. In certain embodiments, this SAbR strategy may be used with TTFields. The radiation therapy can be applied to the tumor before or after delivering the disclosed inhibitors to the tumor. In non-limiting embodiments, the radiation therapy can be simultaneously applied to the tumor with the disclosed inhibitors and/or the TTFields.

The disclosed TTFields can induce DNA damage and slow the repair of IR-induced DNA double strand breaks (DSBs). TTFields exposure can induce replication stress by a decrease in replication fork speed and an increased appearance of R-loop formation with a time of exposure to TTFields. As certain genes associated with replication fork maintenance are also involved in different DNA repair pathways, TTFields treatment can result in both increased replication stress and increased DNA damage because of reduced DNA repair capacity. The disclosed TTFields can cause the development of a conditional vulnerability in cancer cells, rendering them more sensitive to DNA damaging agents and to agents that specifically target key enzymes associated with replication fork maintenance and stability.

The disclosed techniques can induce a conditional vulnerability environment by increasing replication stress in cancer cells. The disclosed inhibitors (e.g., cisplatin, pemetrexed, gemcitabine, and 5-FU) target the replication stress pathway directly or indirectly. Hence using TTFields in combination with such inhibitors can enhance the efficacy of these chemo agents as they both can act on a similar mechanism. For example, TTFields can affect the replication stress pathway through the dysregulation of the E2F family of transcription factors. By determining the role of the E2F family members, the disclosed techniques allow the targeting of specific combinations of TTFields and radiation, chemo or biologic agents directed at proteins ordinarily regulated by E2F signaling. The E2F family dysregulated cells may become vulnerable to the use of targeted agents against DNA repair and/or replication stress. In this case, therapeutic success can be enhanced by the synergistic cell killing seen when the disclosed TTFidelds techniques are combined with the disclosed inhibitors/agents.

### EXPERIMENTAL RESULTS

Using certain embodiments disclosed herein, TTFields in combination with various inhibitors and/or radiation treatment (IR) were applied to NSCLC cells or pancreatic cancer cells. For example, a predetermined inhibitor at a predetermined concentration was delivered to NSCLC cells or pancreatic cancer cells, and the cells were immediately exposed to TTFields (e.g., 100-250 kHz and 1-20 V/cm v/cm) for about 24, 28, or 72 hours. The predetermined concentration was a cell line specific concentration for optimal target cell killing. Then, the cells were irradiated (at a dose of 2Gy) for less than a minute (e.g., 3.25 Gy/min) and immediately plated for survival.

The combinatorial and synergistic effects of TTFields in combination with a PARP inhibitor and/or IR on pancreatic cancer cell survival were evaluated through a cell death/survival analysis assay (i.e., Clonogenic cell survival assay).

FIG. 3 depicts example effects of TTFields, Olaparib, radiation, and a combination thereof on pancreatic cancer cells. In particular, FIG. 3 depicts the effects of TTFields in combination with a PARP inhibitor and/or IR on Panc-1 and 04.03 cell survival (i.e., surviving fraction). As shown in FIG. 3, TTFields, Olaparib (i.e., PARP inhibitor) and IR significantly reduced the surviving fraction of Panc-1 and 04.03 cells. Furthermore, the combinations of TTFields, Olaparib (i.e., PARP inhibitor) and/or IR showed synergistic effects on the Panc-1 and 04.03 cell survival. Olaparib was given concomitant with TTFields. Radiation was given 24, 48 or 72 h after the initiation of TTFields and specific agents. Tables 1 and 2 provide quantification of the synergistic effects on Panc-1 (Table I) and 04.03 cell survival (Table II). Synergistic effects were observed when the combination index (CI) was >1, and the P-value was <0.05 for a given time point and a given cell line. Values greater than 1.0 are considered as synergistic, that is, greater than the sum of the individual responses. The following formulas were used for calculating CI: SF=Survival Fraction; CI (TTFields)+(2Gy)=(SF_{2Gy}×SF_{TTFields})/SF_{2Gy}+TTFields; CI(TTFields)+(Olap)=(SF_{Olap}×SF_{TTFields})/SF_{Olap+TTFields}; CI (2Gy)+(Olap)=(SF_{2Gy}×SF_{Olap})/SF_{2Gy+Olap}; and CI (TTFields)+(2Gy)+(Olap)=(SF_{2Gy}×SF_{Olap}×SF_{TTFields})/SF_{TTFields+2Gy+Olap}. Based on these criteria and the results summarized in Tables I and II, the combined effects of TTFields, IR, and olaparib on pancreatic cancer cell survival/death was found to be synergistic. Based upon the Highest Single Agent (HAS) approach. The calculation for the Combination Index can be found below.

TABLE 1
Combined Effects of TTFields, IR, and Olaparib on Panc-1 cells.

| Time | TTFields + 2Gy | | TTFields + Olap | | 2Gy + Olap | | TTFields + 2Gy + Olap | |
|---|---|---|---|---|---|---|---|---|
| Point | CI | P-value | CI | P-value | CI | P-value | CI | P-value |
| 24 h | 1.075 | 0.04 | 1.035 | 0.01 | 0.890 | 0.037 | 1.589 | 0.016 |
| 48 h | 1.357 | 0.02 | 1.209 | 0.008 | 0.975 | 0.017 | 1.817 | 0.021 |
| 72 h | 1.818 | 0.01 | 1.215 | 0.02 | 1.030 | 0.028 | 2.028 | 0.038 |

TABLE 2
Combined Effects of TTFields, IR, and Olaparib on 04.03 cells.

| Time | TTFields + 2Gy | | TTFields + Olap | | 2Gy + Olap | | TTFields + 2Gy + Olap | |
|---|---|---|---|---|---|---|---|---|
| Point | CI | P-value | CI | P-value | CI | P-value | CI | P-value |
| 24 h | 1.097 | 0.025 | 1.101 | 0.034 | 1.550 | 0.03 | 2.863 | 0.01 |
| 48 h | 1.739 | 0.01 | 0.973 | 0.024 | 1.582 | 0.01 | 2.066 | 0.02 |
| 72 h | 3.324 | 0.003 | 1.042 | 0.006 | 1.462 | 0.01 | 3.818 | 0.002 |

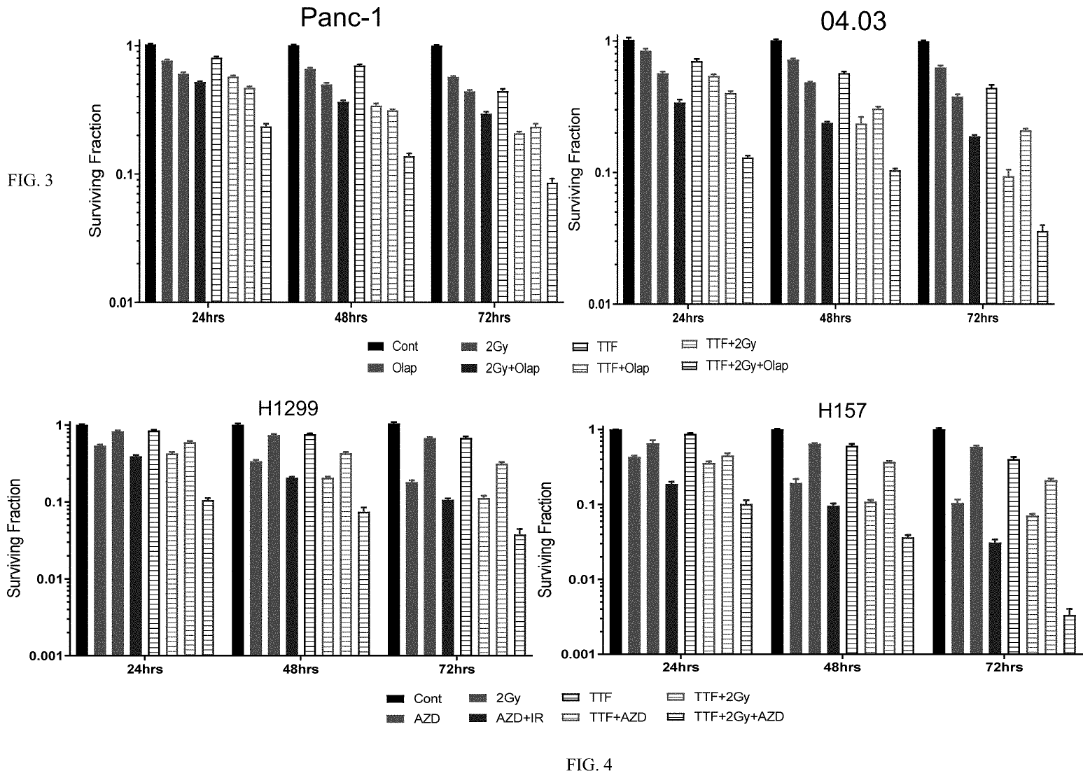

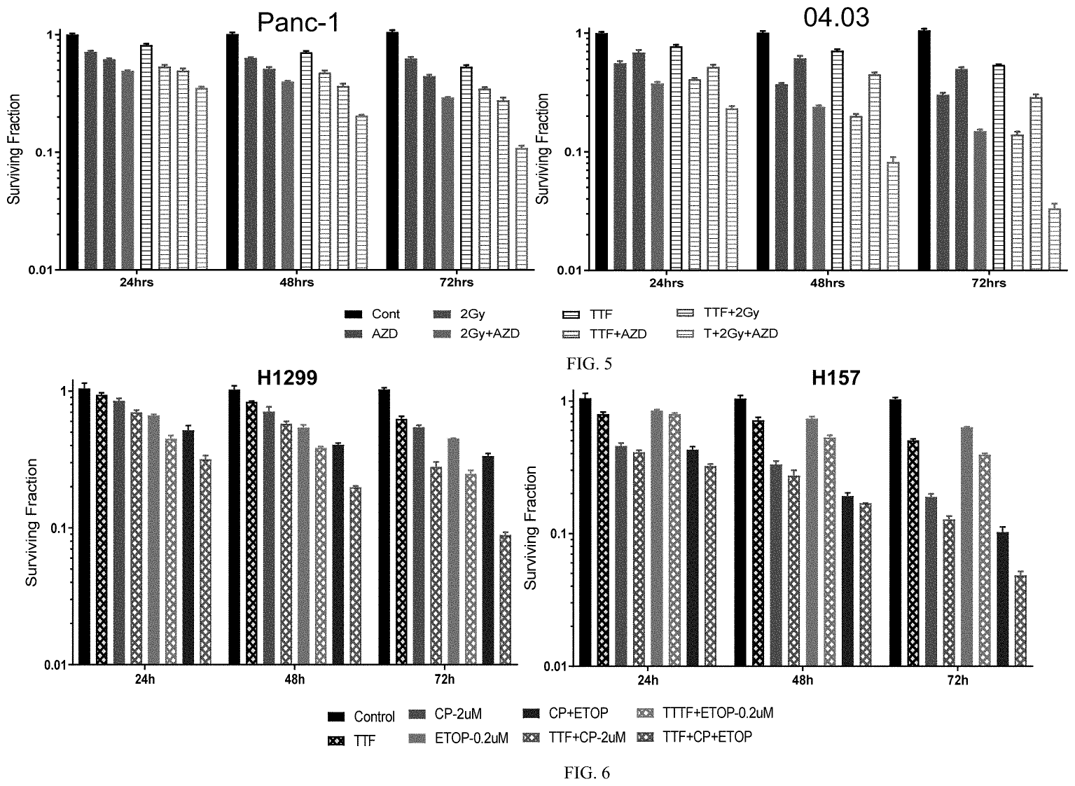

FIGS. 4 and 5 depict example effects of TTFields, AZD6738, radiation, and a combination thereof on non-small cell lung cancer cells (NSCLC) and pancreatic cancer cell, respectively. In particular, FIGS. 4 and 5 show TTFields exposure synergistically increases the effects of ATR inhibitor AZD6738 on NSLCL cells (FIG. 4) survival and pancreatic cancer cell (FIG. 5) survival. TTFields were used together with ATR inhibitor AZD6738 and radiation (IR). A synergistic effect was observed when AZD6738 and IR were combined with TTFields using the highest single agent (HSA) approach. The following formulas were used for calculating CI: SF=Survival Fraction; CI (TTFields)+(2Gy)=(SF_{2Gy}×SF_{TTFields})/SF_{2Gy+TTFields}; CI(TTFields)+(AZD)=(SF_{AZD}×SF_{TTFields})/SF_{AZD+TTFields}; CI (2Gy)+(AZD)=(SF_{2Gy}×SF_{AZD})/SF_{2Gy+AZD}; and CI (TTFields)+(2Gy)+(AZD)=(SF_{2Gy}×SF_{AZD}×SF_{TTFields})/SF_{TTFields+2Gy+AZD}.

As shown in FIG. 4, TTFields, AZD6738 (i.e., ATR inhibitor) and IR significantly reduced the surviving fraction of H1299 and H157 cells. Furthermore, the combinations of TTFields, AZD6738 (i.e., ATR inhibitor) and/or IR showed synergistic effects on H1299 and H157 cell survival.

As shown in FIG. 5, TTFields, AZD6738 (i.e., ATR inhibitor) and IR significantly reduced the surviving fraction of Panc-1 and 04.03 cells. Furthermore, the combinations of TTFields, AZD6738 (i.e., ATR inhibitor) and/or IR showed synergistic effects on Panc-1 and 04.03 cell survival.

Tables 3 and 4 provide quantification of the synergistic effects on H1299 cells (Table 3) and H157 (Table 4). Synergistic effects were observed when the combination index (CI) was >1, and the P-value was <0.05 for a given time point and a given cell line. Based on these criteria and the results summarized in Table 3 and 4, the combined effects of TTFields, IR, and AZD6738 on NSLCL cell survival/death was found to be synergistic. CI values are based upon the time of TTFields and agent exposure and radiation or not. P-values represent two-sided student's T-test for statistical significance.

TABLE 3
Combined Effects of TTFields, IR, and AZD on H1299 cells.

| Time | TTFields + AZD | | TTFields + 2Gy | | AZD + 2Gy | | TTFields + 2Gy + AZD | |
|---|---|---|---|---|---|---|---|---|
| Point | CI | P-value | CI | P-value | CI | P-value | CI | P-value |
| 24 h | 1.070 | 0.02 | 1.172 | 0.01 | 1.127 | 0.02 | 3.553 | 0.02 |
| 48 h | 1.242 | 0.01 | 1.314 | 0.01 | 1.215 | 0.01 | 2.535 | 0.01 |
| 72 h | 1.092 | 0.03 | 1.462 | 0.02 | 1.143 | 0.006 | 2.206 | 0.002 |

TABLE 4
Combined Effects of TTFields, IR, and AZD on H157 cells.

| Time | TTFields + AZD | | TTFields + 2Gy | | AZD + 2Gy | | TTFields + 2Gy + AZD | |
|---|---|---|---|---|---|---|---|---|
| Point | CI | P-value | CI | P-value | CI | P-value | CI | P-value |
| 24 h | 1.048 | 0.01 | 1.275 | 0.02 | 1.498 | 0.03 | 2.430 | 0.01 |
| 48 h | 1.072 | 0.01 | 1.057 | 0.03 | 1.292 | 0.02 | 2.062 | 0.005 |
| 72 h | 1.036 | 0.02 | 1.183 | 0.01 | 3.115 | 0.005 | 4.874 | 0.008 |

Tables 5 and 6 provide quantification of the synergistic effects on Panc-1 cells (Table 5) and 04.03 cells (Table 6). Synergistic effects were observed when the combination index (CI) was >1, and the P-value was <0.05 for a given time point and a given cell line. Based on these criteria and the results summarized in Table 5 and 6, the combined effects of TTFields, IR, and AZD6738 on pancreatic cancer cell survival/death was found to be synergistic.

TABLE 5
Combined Effects of TTFields, IR, and AZD on Panc-1 cells.

| Time | TTFields + AZD | | TTFields + 2Gy | | AZD + 2Gy | | TTFields + 2Gy + AZD | |
|---|---|---|---|---|---|---|---|---|
| Point | CI | P-value | CI | P-value | CI | P-value | CI | P-value |
| 24 h | 1.088 | 0.02 | 1.019 | 0.026 | 0.894 | 0.03 | 1.068 | 0.01 |
| 48 h | 0.970 | 0.018 | 0.991 | 0.04 | 0.879 | 0.027 | 1.153 | 0.02 |
| 72 h | 1.072 | 0.03 | 1.113 | 0.02 | 1.030 | 0.038 | 1.514 | 0.002 |

TABLE 6
Combined Effects of TTFields, IR, and AZD on 04.03 cells.

| Time | TTFields + AZD | | TTFields + 2Gy | | AZD + 2Gy | | TTFields + 2Gy + AZD | |
|---|---|---|---|---|---|---|---|---|
| Point | CI | P-value | CI | P-value | CI | P-value | CI | P-value |
| 24 h | 1.049 | 0.03 | 1.020 | 0.04 | 1.018 | 0.02 | 1.272 | 0.01 |
| 48 h | 1.550 | 0.01 | 1.048 | 0.024 | 1.158 | 0.04 | 2.394 | 0.01 |
| 72 h | 1.287 | 0.02 | 1.025 | 0.04 | 1.011 | 0.03 | 2.674 | 0.008 |

FIG. 6 depicts example effects of TTFields, Cisplatin, Etoposide, and a combination thereof on NSCLC. In particular, FIG. 6 shows TTFields exposure synergistically increases the efficacy of Topoisomerase II inhibitor, etoposide. A synergistic effect can be observed when Etoposide (ETP) is combined with TTFields by using the highest single agent (HSA) approach. CP and ETP were tested alone or in combination and with/without TTFields. As shown in FIG. 6, the combination of TTFields synergistically enhanced the NSCLS cell killing capacity when CP or ETP was used either alone or together.

Tables 7 and 8 provide quantification of the synergistic effects of TTFields in combination with Cisplatin and/or Etoposide on H1299 (Table 7) and H157 cells (Table 8). Synergistic effects were observed when the combination index (CI) was >1, and the P-value was <0.05 for a given time point and a given cell line. The following formulas are used for calculating combination Index: SF=Survival Fraction; CI (TTFields)+(CP)=(SF_{CP}×SF_{TTFields})/SF_{CP+TTFields}; CI (TTFields)+(ETOP)=(SF_{ETOP}×SF_{TTFields})/SF_{ETOP+TTFields}; CI (CP)+(ETOP)=(SF_{CP}×SF_{ETOP})/SF_{CP+ETOP}; and CI (TTFields)+(CP)+(ETOP)=(SF_{CP}×SF_{ETOP}×S_{TTFields})/SF_{TTFields+CP+ETOP+TTFields}.

TABLE 7
Combined Effects of TTFields, CP and ETOP on H1299 cells.

| Time | TTFields + CP | | TTFields + ETOP | | CP + ETOP | | TTFields + CP + ETOP | |
|---|---|---|---|---|---|---|---|---|
| Point | CI | P-value | CI | P-value | CI | P-value | CI | P-value |
| 24 h | 1.140 | 0.015 | 1.387 | 0.014 | 1.087 | 0.024 | 1.666 | 0.011 |
| 48 h | 1.021 | 0.012 | 1.174 | 0.004 | 0.947 | 0.007 | 1.612 | 0.002 |
| 72 h | 1.225 | 0.013 | 1.140 | 0.009 | 0.730 | 0.008 | 1.734 | 0.002 |

TABLE 8
Combined Effects of TTFields, CP and ETOP on H157 cells.

| Time | TTFields + CP | | TTFields + ETOP | | CP + ETOP | | TTFields + CP + ETOP | |
|---|---|---|---|---|---|---|---|---|
| Point | CI | P-value | CI | P-value | CI | P-value | CI | P-value |
| 24 h | 0.887 | 0.01 | 0.842 | 0.02 | 0.899 | 0.02 | 0.953 | 0.01 |
| 48 h | 0.869 | 0.02 | 0.995 | 0.01 | 1.275 | 0.009 | 1.035 | 0.02 |
| 72 h | 1.079 | 0.006 | 1.021 | 0.003 | 1.362 | 0.005 | 1.746 | 0.001 |

Based on these criteria and the results summarized in Table 7 and 8, the combined effects of TTFields, CP, and ETOP on NSCLC cell survival/death was found to be synergistic.

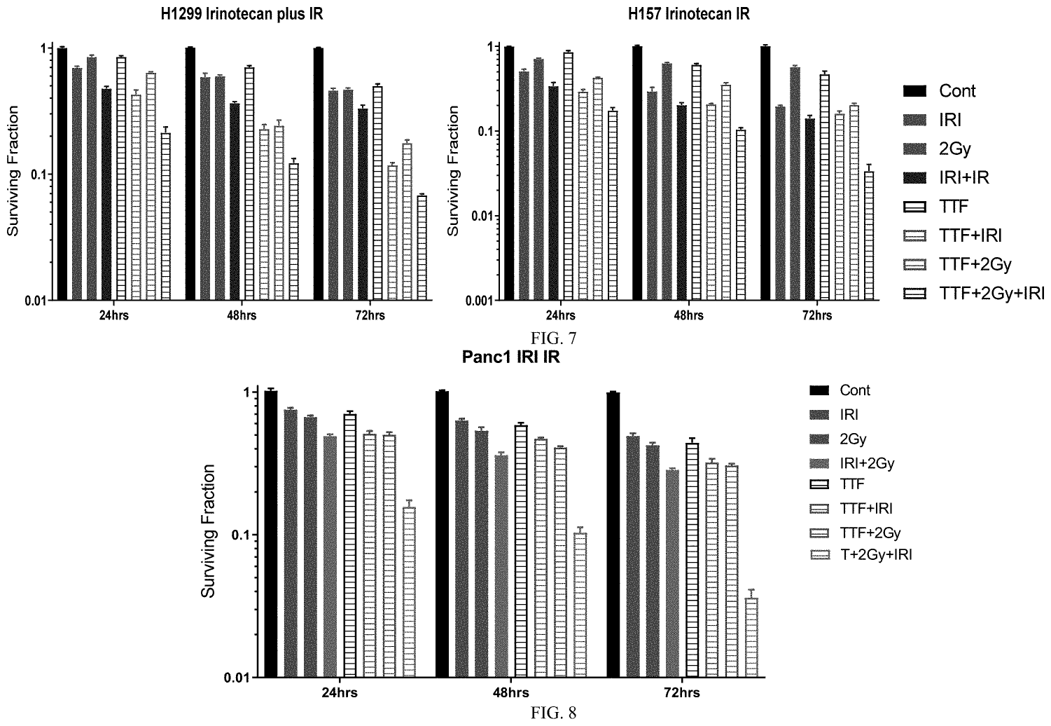

FIG. 7 depicts example effects of TTFields, Irinotecan, radiation, and a combination thereof on NSCLC. In particular, FIG. 7 depicts the effects of combinatorial and synergistic effects of TTFields in combination with irinotecan and/or IR on H1299 and H157 cell survival (i.e., surviving fraction). As shown in FIG. 7, TTFields, Irinotecan and IR significantly reduced the surviving fraction of H1299 and H157 cells. Furthermore, the combinations of TTFields, Irinotecan and/or IR showed synergistic effects on H1299 and H157 cell survival.

Tables 9 and 10 provide quantification of the synergistic effects of TTFields in combination with irinotecan and/or IR on H1299 survival (Table 9) and H157 cell survival (Table 10). Synergistic effects were observed when the combination index (CI) was >1, and the P-value was <0.05 for a given time point and a given cell line. The following formulas are used for calculating the combination Index: SF=Survival Fraction; CI (TTFields)+(2Gy)=(SF_{2Gy}×SF_{TTFields})/SF_{2Gy+TTFields}; CI(TTFields)+(Irino)=(SF_{Irino}×SF_{TTFields})/SF_{Irino+TTFields}; CI (2Gy)+(Irino)=(S_{2Gy}×SF_{Irino})SF_{2Gy+Irino}; and CI (TTFields)+(2Gy)+(Irino)=(SF_{2Gy}×SF_{Irino}×SF_{TTFields})/SF_{TTFields+2Gy+Irino}.

TABLE 9
Combined Effects of TTFields, Irinotecan, and IR on H1299 cells.

| Time | TTFields + Irino | | TTFields + 2Gy | | Irino + 2Gy | | TTFields + Irino + 2Gy | |
|---|---|---|---|---|---|---|---|---|
| Point | CI | P-value | CI | P-value | CI | P-value | CI | P-value |
| 24 h | 1.388 | 0.017 | 1.134 | 0.011 | 1.244 | 0.024 | 2.365 | 0.031 |
| 48 h | 1.821 | 0.022 | 1.827 | 0.024 | 1.012 | 0.017 | 2.130 | 0.011 |
| 72 h | 2.081 | 0.035 | 1.526 | 0.012 | 1.027 | 0.009 | 2.051 | 0.010 |

TABLE 10
Combined Effects of TTFields, Irinotecan and, IR on H157 cells.

| Time | TTFields + Irino | | TTFields + 2Gy | | Irino + 2Gy | | TTFields + Irino + 2Gy | |
|---|---|---|---|---|---|---|---|---|
| Point | CI | P-value | CI | P-value | CI | P-value | CI | P-value |
| 24 h | 1.483 | 0.031 | 1.433 | 0.011 | 1.064 | 0.014 | 1.763 | 0.021 |
| 48 h | 1.174 | 0.020 | 1.074 | 0.028 | 1.244 | 0.015 | 1.463 | 0.011 |
| 72 h | 1.136 | 0.020 | 1.314 | 0.019 | 1.275 | 0.018 | 2.501 | 0.024 |

Based on these criteria and the results summarized in Table 9 and 10, the combined effects of TTFields, Irinotecan, and/or IR on NSCLC cell survival/death was found to be synergistic.

FIG. 8 depicts example effects of TTFields, Irinotecan, radiation, and a combination thereof on pancreatic cancer cells. In particular, FIG. 8 depicts the effects of combinatorial and synergistic effects of TTFields in combination with irinotecan and/or IR on Panc-1 cell survival (i.e., surviving fraction). As shown in FIG. 8, TTFields, Irinotecan and IR significantly reduced the surviving fraction of Panc-1 cell survival. Furthermore, the combinations of TTFields, Irinotecan and/or IR showed synergistic effects on Panc-1 cell survival.

Table 11 provides quantification of the synergistic effects of TTFields in combination with irinotecan and/or IR on Panc-1 cell survival. Synergistic effects were observed when the combination index (CI) was >1, and the P-value was <0.05 for a given time point and a given cell line. The following formulas are used for calculating the combination

TABLE 11
Combined Effects of TTFields, Irinotecan, and IR on Pan-1 cells.

| Time | TTFields + Irino | | TTFields + 2Gy | | Irino + 2Gy | | TTFields + Irino + 2Gy | |
|---|---|---|---|---|---|---|---|---|
| Point | CI | P-value | CI | P-value | CI | P-value | CI | P-value |
| 24 h | 1.082 | 0.012 | 0.979 | 0.013 | 1.022 | 0.014 | 2.360 | 0.012 |
| 48 h | 0.974 | 0.017 | 0.934 | 0.014 | 1.009 | 0.027 | 2.335 | 0.008 |
| 72 h | 1.030 | 0.015 | 1.013 | 0.032 | 1.132 | 0.018 | 4.454 | 0.003 |

Based on these criteria and the results summarized in Table 11, the combined effects of the combination of TTFields together with irinotecan, which traps topoisomerase I-DNA in ternary cleavage complex on NSCLC cell survival/death was found to be synergistic.

Although the degree of sensitization and synergy may vary across cell lines, TTFields increased cell killing efficacy of etoposide synergistically, which forms a ternary complex with topoisomerase II and prevents re-ligation of DNA strands to elicit DNA strand breaks and induce replication stress.

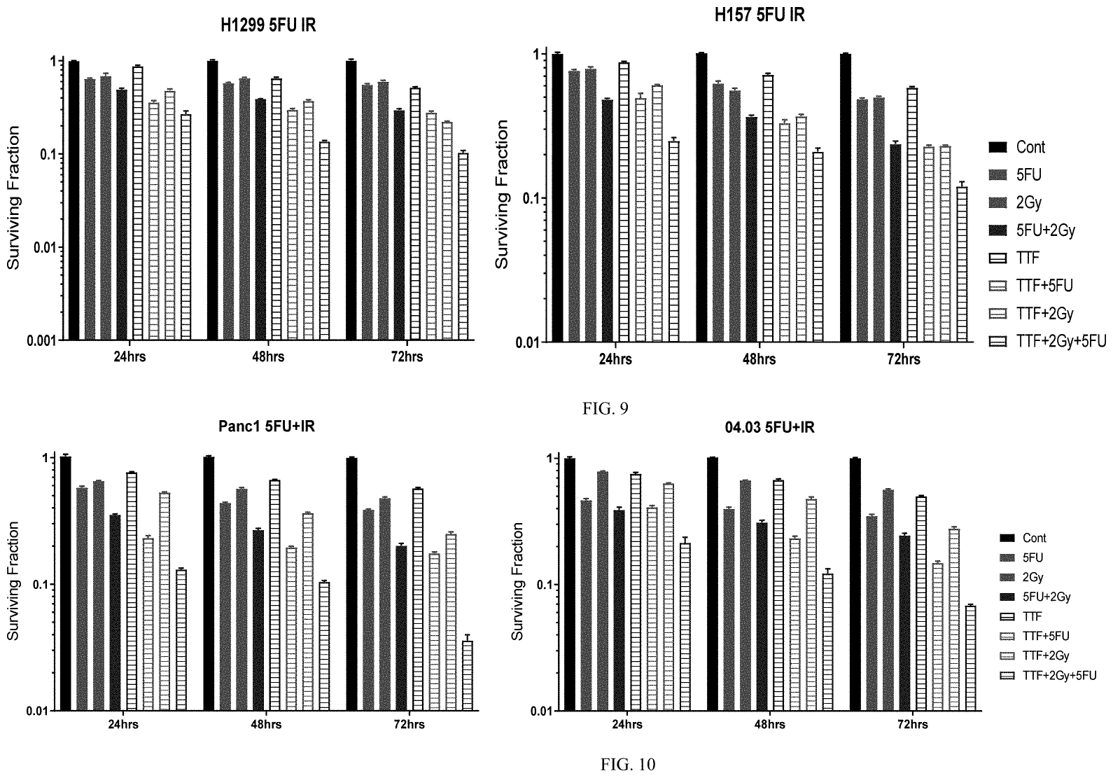

FIGS. 9 and 10 depict example effects of TTFields, 5-FU, radiation, and a combination thereof on NSCLC and pancreatic cancer cells, respectively. In particular, FIGS. 9 and 10 show TTFields exposure synergistically increases the effects of the combination with 5-Fluorouracil (FU) and/or IR on NSCLC cell survival (FIG. 9) and pancreatic cancer cells (FIG. 10). As shown in FIG. 9, TTFields, 5-FU, and IR significantly reduced the surviving fraction of H1299 and H157 cell survival. Furthermore, the combinations of TTFields, 5-FU, and/or IR showed synergistic effects on H1299 and H157 cell survival. FIG. 10 shows that TTFields, 5-FU, and IR significantly reduced the surviving fraction of Panc-1 and 04.03 cell survival. Furthermore, the combinations of TTFields, 5-FU, and/or IR showed synergistic effects on Panc-1 and 04.03 cell survival.

Tables 12 and 13 provide quantification of the synergistic effects of TTFields in combination with 5-Fluorouracil (FU), and/or IR on H1299 survival (Table 12) and H157 cell survival (Table 13). Synergistic effects were observed when the combination index (CI) was >1, and the P-value was <0.005 for a given time point and a given cell line. The following formulas are used for calculating the combination Index: SF=Survival Fraction; CI (TTFields)+(2Gy)=(SF_{2Gy}×SF_{TTFields})/SF_{2Gy+TTFields}; CI(TTFields)+(5FU)=(SF_{5FU}×SF_{TTFields})/SF_{5FU+TTFields}; CI (2Gy)+(5FU)=(SF_{2Gy}×SF_{5FU})/SF_{2Gy+5FU}; and CI (TTFields)+(2Gy)+(5FU)=(SF_{2Gy}×SF_{5FU}×SF_{TTFields})/SF_{TTFields+2Gy+5FU}.

TABLE 12
Combined Effects of TTFields, -Fluorouracil (FU), and/or IR on H1299 cells.

| Time | TTFields + 5FU | | TTFields + 2Gy | | 5FU + 2Gy | | TTFields + 5FU + 2Gy | |
|---|---|---|---|---|---|---|---|---|
| Point | CI | P-value | CI | P-value | CI | P-value | CI | P-value |
| 24 h | 1.352 | 0.017 | 1.138 | 0.011 | 1.254 | 0.034 | 1.762 | 0.015 |
| 48 h | 1.348 | 0.020 | 1.085 | 0.024 | 0.950 | 0.017 | 1.263 | 0.023 |
| 72 h | 1.326 | 0.035 | 1.287 | 0.026 | 1.042 | 0.028 | 1.345 | 0.014 |

TABLE 13
Combined Effects of -Fluorouracil (FU), and/or IR on H157 cells.

| Time | TTFields + 5FU | | TTFields + 2Gy | | 5FU + 2Gy | | TTFields + 5FU + 2Gy | |
|---|---|---|---|---|---|---|---|---|
| Point | CI | P-value | CI | P-value | CI | P-value | CI | P-value |
| 24 h | 1.559 | 0.019 | 1.255 | 0.013 | 0.894 | 0.014 | 1.433 | 0.015 |
| 48 h | 1.252 | 0.020 | 1.142 | 0.028 | 0.962 | 0.017 | 1.783 | 0.008 |
| 72 h | 1.106 | 0.037 | 1.383 | 0.019 | 1.125 | 0.010 | 1.658 | 0.008 |

Based on these criteria and the results summarized in Tables 12 and 13, the combined effects of TTFields, 5-Fluorouracil (FU), and/or IR on NSCLC cell survival/death was found to be synergistic.

Tables 14 and 15 provide quantification of the synergistic effects of TTFields in combination with 5-Fluorouracil (FU), and/or IR on Panc-1 survival (Table 14) and 04.03 cell survival (Table 15). Synergistic effects were observed when the combination index (CI) was >1, and the P-value was <0.05 for a given time point and a given cell line.

TABLE E
Combined Effects of TTFields, -Fluorouracil (FU), and/or IR on Panc-1 cells.

| Time | TTFields + 5FU | | TTFields + 2Gy | | 5FU + 2Gy | | TTFields + 5FU + 2Gy | |
|---|---|---|---|---|---|---|---|---|
| Point | CI | P-value | CI | P-value | CI | P-value | CI | P-value |
| 24 h | 1.896 | 0.019 | 0.942 | 0.013 | 1.064 | 0.014 | 2.198 | 0.015 |
| 48 h | 1.492 | 0.020 | 1.029 | 0.028 | 0.943 | 0.017 | 1.572 | 0.008 |
| 72 h | 1.276 | 0.037 | 1.143 | 0.019 | 1.028 | 0.010 | 2.999 | 0.008 |

TABLE 15
Combined Effects of -Fluorouracil (FU), and/or IR on 04.03 cells.

| Time | TTFields + 5FU | | TTFields + 2Gy | | 5FU + 2Gy | | TTFields + 5FU + 2Gy | |
|---|---|---|---|---|---|---|---|---|
| Point | CI | P-value | CI | P-value | CI | P-value | CI | P-value |
| 24 h | 0.855 | 0.016 | 0.933 | 0.023 | 0.936 | 0.014 | 1.281 | 0.019 |
| 48 h | 1.202 | 0.012 | 0.940 | 0.014 | 0.896 | 0.017 | 1.519 | 0.009 |
| 72 h | 1.268 | 0.017 | 1.119 | 0.012 | 1.039 | 0.010 | 1.632 | 0.005 |

Based on these criteria and the results summarized in Tables 13 and 14, the combined effects of TTFields, 5-Fluorouracil (FU), and/or IR on pancreatic cancer cell survival/death was found to be synergistic.

### EXEMPLARY APPARATUSES

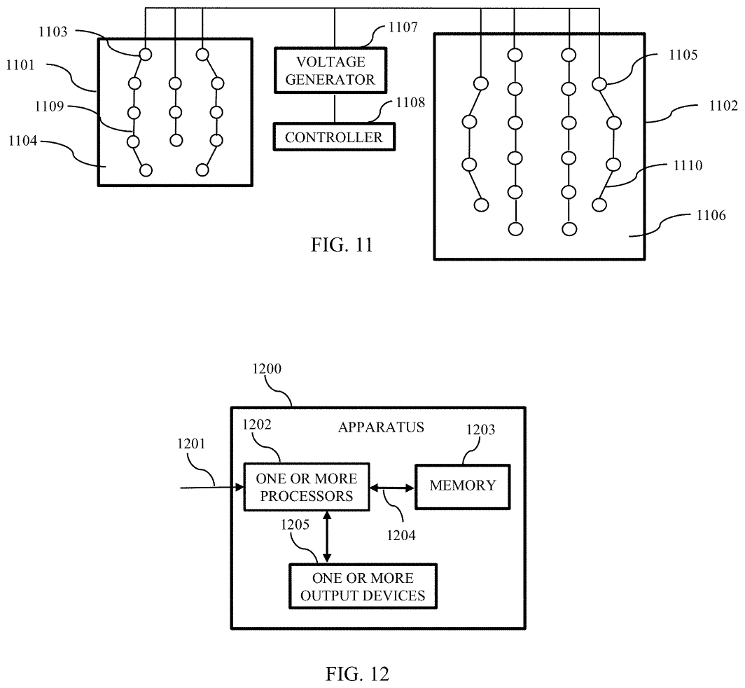

FIG. 11 depicts one example of an apparatus to apply TTFields with modulated electric fields to a subject's body. The first transducer 1101 includes 13 electrode elements 1103, which are positioned on the substrate 1104, and the electrode elements 1103 are electrically and mechanically connected to one another through a conductive wiring 1109. The second transducer 1102 includes 20 electrode elements 1105, which are positioned on the substrate 1106, and the electrode elements 1105 are electrically and mechanically connected to one another through a conductive wiring 1110. The first transducer 1101 and the second transducer 1102 are connected to an AC voltage generator 1107 and a controller 1108. The controller 1108 may include one or more processors and memory accessible by the one or more processors. The memory may store instructions that when executed by the one or more processors, control the AC voltage generator 1107 to implement one or more embodiments of the invention. In some embodiments, the AC voltage generator 1107 and the controller 1108 may be integrated in the first transducer 1101 and the second transducer 1102 and form a first electric field generator and a second electric field generator.

FIG. 12 depicts an example computer apparatus for use with the embodiments herein. As an example, the apparatus 1200 may be a computer to implement certain inventive techniques disclosed herein. As an example, the apparatus 1200 may be a controller apparatus to apply TTFields with modulated electric fields for the embodiments herein. The controller apparatus 1200 may be used as the controller 1108 of FIG. 11. The apparatus 1200 may include one or more processors 1202, one or more output devices 1205, and a memory 1203.

In one example, based on input 1201, the one or more processors generate control signals to control the voltage generator to implement an embodiment of the invention. In one example, the input 1201 is user input. In another example, the input 1201 may be from another computer in communication with the controller apparatus 1200. The output devices 1205 may provide the status of the operation of the invention, such as transducer selection, voltages being generated, and other operational information. The output devices 1205 may provide visualization data according to certain embodiments of the invention.

The memory 1203 is accessible by the one or more processors 1202 via the link 1104 so that the one or more processors 1202 can read information from and write information to the memory 1203. The memory 1203 may store instructions that when executed by the one or more processors 1202 implement one or more embodiments of the invention.

### ILLUSTRATIVE EMBODIMENTS

The invention includes other illustrative embodiments, such as the following.

Illustrative Embodiment 1. A method of treating a tumor in a subject, comprising: delivering an ATR inhibitor to the tumor, and applying a tumor treating field to the tumor at a frequency between approximately 50 kHz and approximately 1,000 kHz.

Illustrative Embodiment 2. The method of Illustrative Embodiment 1, wherein an intensity of the tumor treating field is between approximately 1 V/cm and approximately 4 V/cm, wherein the frequency of the tumor treating field is between approximately 150 kHZ and approximately 250 kHZ, wherein the tumor comprises a glioblastoma cancer cell.

Illustrative Embodiment 3. The method of Illustrative Embodiment 1, wherein the ATR inhibitor comprises Ceralasertib (AZD6738) in an amount between approximately 1 μM and approximately 50 μM.

Illustrative Embodiment 4. The method of Illustrative Embodiment 1, wherein the ATR inhibitor is delivered through infusion.

Illustrative Embodiment 5. The method of Illustrative Embodiment 1, wherein the ATR inhibitor is delivered in a cocktail form.

Illustrative Embodiment 6. The method of Illustrative Embodiment 1 comprises delivering a radiation therapy to the tumor.

Illustrative Embodiment 7. The method of Illustrative Embodiment 6, wherein a dose of the radiation therapy is between approximately 1 Gy to approximately 18 Gy.

Illustrative Embodiment 8. A method of treating a tumor in a subject, comprising: delivering an ATR inhibitor or one or more of DNA replication stress inducing agent; delivering at least one of an E2F inhibitor, a CDK4/6 inhibitor, a PARP inhibitor, or a platinum compound to the tumor; delivering a radiation therapy to the tumor; applying tumor treating fields to the tumor at a frequency between approximately 50 kHz and approximately 1,000 kHz.

Illustrative Embodiment 9. A method of treating a tumor in a subject, comprising: delivering an ATR inhibitor and one or more of DNA replication stress inducing agent to the tumor; delivering a radiation therapy to the tumor; applying a tumor treating field to the tumor at a frequency between approximately 50 kHz and approximately 1,000 kHz.

Embodiments illustrated under any heading or in any portion of the disclosure may be combined with embodiments illustrated under the same or any other heading or other portion of the disclosure unless otherwise indicated herein or otherwise clearly contradicted by context.

Numerous modifications, alterations, and changes to the described embodiments are possible without departing from the scope of the present invention defined in the claims. It is intended that the present invention not be limited to the described embodiments but that it has the full scope defined by the language of the following claims and equivalents thereof.

What is claimed is:

1. A method of treating a tumor in a subject, comprising:
delivering an ATR inhibitor to the tumor; and
applying a tumor treating field to the tumor at a frequency between approximately 50 kHz and approximately 1,000 kHz.

2. The method of claim 1, wherein the ATR inhibitor comprises at least one of Schisandrin B, Nu6027, Dactolisib, EPT-46464, VE-821, AZ20, Berzosertib, Torin-2, Ceralasertib (AZD6738), Tetrahydropyrazolo [1,5-a]pyrazines, Azabenzimidazoles, Gartisertib, (M4344 or VX-803), Bayl895344 (Elimsuretib), CGK 733, RP-3500, ATR-IN-4, VE-821, AZ20, ETP-46464, or ATR inhibitor 1.

3. The method of claim 1, wherein the tumor comprises at least one a lung cancer cell, a breast cancer cell, a pancreatic cancer cell, a glioblastoma cell, a prostate cancer cell, a liver cancer cell, a fallopian tube cancer cell, a peritoneal cancer cell, a skin cancer cell, a cervical cancer cell, or an ovarian cancer cell.

4. The method of claim 1, wherein an intensity of the tumor treating field is between approximately 1 V/cm and approximately 4 V/cm.

5. The method of claim 1, wherein the frequency of the tumor treating field is between approximately 100 kHZ and approximately 500 kHZ.

6. The method of claim 1, wherein the frequency of the tumor treating field is approximately 100 kHZ, approximately 150 kHZ, approximately 200 kHZ, or approximately 250 kHZ.

7. The method of claim 1, wherein at least a portion of the applying step is performed simultaneously with at least a portion of the delivering step.

8. The method of claim 1, further comprising delivering a DNA replication stress inducing agent to the tumor.

9. The method of claim 8, wherein the DNA replication stress inducing agent comprises at least one of a platinum compound, an alkylating agent, a weel inhibitor, a Chk1 inhibitor, a thymidylate synthase inhibitor, a ribonucleotide reductase inhibitor, Topoisomerase I inhibitor, Topoisomerase II inhibitor, a maternal embryonic leucine zipper kinase (MELK) inhibitor, or a NEDD8-activating enzyme (NAE) inhibitor.

10. The method of claim 9, wherein if the DNA replication stress inducing agent delivered to the tumor is the platinum compound, the platinum compound comprises at least one of cisplatin, carboplatin, oxaliplatin, dicycoplatin, or lipoplatin,
wherein if the DNA replication stress inducing agent delivered to the tumor is the alkylating agent, the alkylating agent comprises at least one of cyclophosphamide or temozolomide,
wherein if the DNA replication stress inducing agent delivered to the tumor is the weel inhibitor, the weel inhibitor comprises at least one of Adavosertib-MK1775 or PD0166285,
wherein if the DNA replication stress inducing agent delivered to the tumor is the Chk1 inhibitor, the Chk1 inhibitor comprises at least one of UCN-01, LY2606368, SAR-020106, AZD7762, or PD0166285,
wherein if the DNA replication stress inducing agent delivered to the tumor is the thymidylate synthase inhibitor, the thymidylate synthase inhibitor comprises at least one of 5-FU or pemetrexed,
wherein if the DNA replication stress inducing agent delivered to the tumor is the ribonucleotide reductase inhibitor, the ribonucleotide reductase inhibitor comprises gemcitabine,
wherein if the DNA replication stress inducing agent delivered to the tumor is the Topoisomerase I inhibitor, the Topoisomerase I inhibitor comprises at least one of Irinotecan or Topotecan,
wherein if the DNA replication stress inducing agent delivered to the tumor is the Topoisomerase II inhibitor, the Topoisomerase II inhibitor comprises at least one of etoposide or doxorubicin,
wherein if the DNA replication stress inducing agent delivered to the tumor is the MELK inhibitor, the MELK inhibitor comprises OTS167, and
wherein if the DNA replication stress inducing agent delivered to the tumor is the NAE inhibitor, the NAE inhibitor comprises MLN4924.

11. The method of claim 8, further comprising delivering an additional DNA replication stress inducing agent to the tumor.

12. The method of claim 1, further comprising delivering at least one of an E2F inhibitor, a CDK4/6 inhibitor, or a PARP inhibitor.

13. The method of claim 12, wherein the E2F inhibitor is delivered to the tumor, and wherein the E2F inhibitor is HLM006474.

14. The method of claim 13, wherein the CDK4/6 inhibitor is delivered to the tumor, and wherein the CDK4/6 inhibitor is abemaciclib, palbociclib, ribociclib, or Trilaciclib.

15. The method of claim 12, wherein the PARP inhibitor is delivered to the tumor, and wherein the PARP inhibitor is olaparib, talazoparib, veliparib, rucaparib, BYK204165, niraparib (MK-4827), niraparib (MK-4827), tosylate, or Iniparib.

16. The method of claim 1, further comprising:
delivering a radiation therapy to the tumor.

17. The method of claim 16, wherein the radiation therapy is delivered before or after the tumor treating field is applied.

18. A method of preventing/reducing proliferation of a cell, comprising:
delivering at least one DNA replication stress inducing agent to the cell, wherein the DNA replication stress inducing agent comprises an ATR inhibitor; and
applying a tumor treating field to the cell at a frequency between approximately 50 kHz and approximately 1,000 kHz.

19. A method of treating a tumor in a subject, comprising:
delivering at least two DNA replication stress inducing agents to the tumor, wherein at least one of the DNA replication stress inducing agents comprises an ATR inhibitor;
delivering a radiation therapy to the tumor; and
applying tumor treating fields to the tumor at a frequency between approximately 50 kHz and approximately 1,000 kHz.

20. The method of claim 19, further comprising delivering at least one of an E2F inhibitor, a CDK4/6 inhibitor, a PARP inhibitor, or a platinum compound to the tumor.
